(19) United States
(12) Patent Application Publication
(10) Pub. No.: US 2024/0325467 A1
(43) Pub. Date: Oct. 3, 2024

(54) NUCLEOTIDE PROBIOTIC CAPSULE FOR DELAYING AGING

(71) Applicant: Yusong CHEN, Dalian, Liaoning (CN)

(72) Inventors: Yong LI, Dalian, Liaoning (CN); Ting DING, Dalian, Liaoning (CN); Yusong CHEN, Dalian, Liaoning (CN)

(21) Appl. No.: 18/291,310

(22) PCT Filed: Jun. 10, 2022
(86) PCT No.: PCT/CN2022/098178

§ 371 Date: Jan. 23, 2024

(30) Foreign Application Priority Data
Jul. 30, 2021 (CN) 202110874599.1

(51) Int. Cl. A61K 35/747; A61K 9/48; A61K 31/7068; A61K 31/7072; A61K 31/7076; A61K 31/708; A61K 35/00; A61K 35/744; A61P 39/06

(52) U.S. Cl. CPC A61K 35/747; A61K 9/4816; A61K 9/4825; A61K 9/4833; A61K 31/7068; A61K 31/7072; A61K 31/7076; A61K 31/708; A61K 35/744; A61P 39/06; A61K 2035/115

(57) ABSTRACT

A nucleotide probiotic capsule for delaying aging has a capsule shell and contents. The contents include four exogenous nucleotides and five probiotics. The exogenous nucleotides include 5′-cytidine monophosphate (5′-CMP), 5′-adenosine monophosphate (5′-AMP), 5′-disodium uridylate (5′-UMPNa₂) and 5′-disodium guanylate (5′-GMPNa₂), and the probiotics include Lactobacillus acidophilus, Lactobacillus plantarum, Lactobacillus casei, etc. Each of the probiotics has a content greater than 1×10⁷ CFU/g. The nucleotide probiotic capsule capable of scavenging free radicals in the body, inhibiting oxidative damage of the free radicals to the body, and prolonging the cell life.

### TECHNICAL FIELD

The present invention relates to the field of medicine and health care, in particular to a nucleotide probiotic capsule for delaying aging.

### BACKGROUND

Aging refers to the process in which, after human development and maturity, with the increase of time, tissue and structure of human body changes, function, adaptability and resistance decline, and human tends to die. As a change in complex life phenomenon, aging is a spontaneous and inevitable process. With the improvement of people's living standards, China is about to enter an aging society. Delaying aging and living a long and healthy life are the goals pursued by many people, especially women. The pursuit of longevity has been the dream of human beings for thousands of years. Nowadays, prevention and postponement of aging has become a hot issue in current research. Studies have found that aging is a result of comprehensive effects of various factors such as genetic genes, free radicals, immune dysfunction and the like. Although aging is an irresistible natural law in everyone's life activities, adopting good health care measures and taking anti-aging products can effectively delay aging, delay sickness and death, improve the quality of life, and allow human beings to achieve maximum longevity under existing conditions.

Nucleotides are important components of cells and are precursors of DNA and RNA, which play an important role in cell metabolism, protein synthesis, cell division and the like. Nucleotides become the “essential nutrients” for the body under some specific conditions, such as disease, rapid growth and development, limited nutrient intake, and hindered endogenous synthesis and expression. Therefore, nucleotides can be used as potential substances for delaying aging and prolonging life.

Aging of the body will be accompanied by a variety of chronic diseases, and many of these chronic diseases are related to the imbalance of intestinal flora. Therefore, intestinal flora is also an important indicator affecting body aging. Probiotics are active microorganisms that colonize the human body and play an important role in human health. Studies have found that probiotics have important effects in regulating the balance of intestinal flora, promoting nutrient absorption, and maintaining intestinal health.

At present, there are many products for delaying aging on the market, but these products have low, slow and poor effect. Therefore, it is an important topic to study and develop a product with safe and effective components, non-toxic side effects, and efficient ability to delay body aging.

### SUMMARY OF THE INVENTION

For the above problems, an object of the present invention is to provide a nucleotide probiotic capsule for delaying aging, including four exogenous nucleotides and five probiotics. The nucleotide probiotic capsule has safe and effective components and no-toxic side effects, and has the effects of enhancing immunity, delaying body aging, and enhancing cell vitality.

In order to realize the object of the present invention, the present invention adopts the following technical solutions:

A nucleotide probiotic capsule for delaying aging includes an elliptical capsule shell and contents. The contents includes a mixture of four exogenous 5′-nucleotides or sodium salts thereof and five probiotics, with a weight ratio of 45-75 parts of the 5′-nucleotide mixture and 20-30 parts of probiotics.

Preferably, weight percentages of the four exogenous 5′-nucleotides in the contents are as follows: 15-45% of CMP, 15-55% of AMP, 6-30% of UMP, and 14-35% of GMP.

Preferably, mass percentages of the various probiotics are as follows: 17-32% of Lactobacillus acidophilus, 17-32% of Lactobacillus plantarum, 17-32% of Lactobacillus casei, 10-23% of Lactobacillus helveticus, and 7-19% of Pediococcus pentosaceus.

Preferably, the weight ratios of the contents are as follows: 10-20 parts of 5′-cytidine monophosphate (5′-CMP), 20-30 parts of 5′-adenosine monophosphate (5′-AMP), 5-10 parts of 5′-disodium uridylate (5′-UMPNa₂), 10-15 parts of 5′-disodium guanylate (5′-GMPNa₂), 5-7 parts of Lactobacillus acidophilus, 5-7 parts of Lactobacillus plantarum, 5-7 parts of Lactobacillus casei, 3-5 parts of Lactobacillus helveticus, and 2-4 parts of Pediococcus pentosaceus.

Preferably, the weight ratios of the contents are as follows: 10 parts of 5′-cytidine monophosphate, 20 parts of 5′-adenosine monophosphate, 5 parts of 5′-disodium uridylate, 10 parts of 5′-disodium guanylate, 5 parts of Lactobacillus acidophilus, 5 parts of Lactobacillus plantarum, 5 parts of Lactobacillus casei, 3 parts of Lactobacillus helveticus, and 2 parts of Pediococcus pentosaceus.

Preferably, the weight ratios of the contents are as follows: 15 parts of 5′-cytidine monophosphate, 25 parts of 5′-adenosine monophosphate, 8 parts of 5′-disodium uridylate, 10 parts of 5′-disodium guanylate, 6 parts of Lactobacillus acidophilus, 6 parts of Lactobacillus plantarum, 6 parts of Lactobacillus casei, 4 parts of Lactobacillus helveticus, and 3 parts of Pediococcus pentosaceus.

Preferably, the weight ratios of the contents are as follows: 20 parts of 5′-cytidine monophosphate, 30 parts of 5′-adenosine monophosphate, 10 parts of 5′-disodium uridylate, 15 parts of 5′-disodium guanylate, 7 parts of Lactobacillus acidophilus, 7 parts of Lactobacillus plantarum, 7 parts of Lactobacillus casei, 5 parts of Lactobacillus helveticus, and 4 parts of Pediococcus pentosaceus.

Preferably, a viable bacteria count of each of the probiotics is greater than 1×10⁷ CFU/g.

Preferably, a material of the capsule shell is maltodextrin and soy protein isolate.

Preferably, a weight ratio of the maltodextrin to the soy protein isolate is 1:1-1:2.

Preferably, a weight ratio of the maltodextrin to the soy protein isolate is 1:1.5-1:2.

Preferably, the nucleotides include natural nucleotides and synthetic nucleotides.

Preferably, the probiotics are lyophilized probiotic live bacteria powder.

Another aspect of the present invention provides a method for preparing the nucleotide probiotic capsule. The method includes weighing the nucleotides and probiotics according to the ratio, then uniformly mixing them to obtain a mixed powder, and loading the mixed powder into a capsule shell to obtain the nucleotide probiotic capsule.

Another aspect of the present invention provides an application of the nucleotide probiotic capsule in preparing health care foods and/or drugs for delaying aging.

Compared with the prior art, the present invention has the following beneficial effects:

1. The present invention uses dietary nucleotides and probiotics as raw materials, and the components are safe and effective and have no toxic side effects. When taken together, the nucleotides and probiotics have a synergistic effect, can scavenge free radicals in the body, inhibit oxidative damage of free radicals to the body, prolong cell life, and have various health care effects such as delaying aging, prolonging life, improving immunity, regulating intestinal flora and the like. The nucleotide probiotic capsule of the present invention is convenient to carry and take, and is a health care food suitable for middle-aged and elderly people.

2. The preparation process of the nucleotide probiotic capsule of the present invention is simple and easy to operate, has good stability, and is beneficial to health when taken for a long-term.

### DETAILED DESCRIPTION OF PREFERRED EMBODIMENTS

The present invention is further described by the following embodiments, but is not therefore limited to the scope of the embodiments. In the following embodiments, unless otherwise specified, the experimental methods are conventional methods, and the materials and reagents used can be commercially obtained from biological or chemical companies.

The nucleotide probiotic capsule for delaying aging of the present invention includes an elliptical capsule skin and contents. The compositions of the capsule skin include maltodextrin and a soy isolate protein; and the contents include four dietary nucleotides and multiple probiotics.

### Example 1

A nucleotide probiotic capsule for delaying aging included an elliptical capsule shell and contents. The material of the capsule shell was maltodextrin and soy protein isolate with a weight ratio of 1:1. The components of the contents included a mixture of four 5′-mononucleotides or sodium salts thereof and five lyophilized probiotic live bacteria powder, with ratios according to parts by weight as follows: 10 parts of 5′-cytidine monophosphate, 20 parts of 5′-adenosine monophosphate, 5 parts of 5′-disodium uridylate, 10 parts of 5′-disodium guanylate, 5 parts of Lactobacillus acidophilus, 5 parts of Lactobacillus plantarum, 5 parts of Lactobacillus casei, 3 parts of Lactobacillus helveticus, and 2 parts of Pediococcus pentosaceus. The preparation method comprises the following steps: the above-mentioned contents were uniformly mixed according to the ratios to obtain a mixed powder, the mixed powder was loaded into capsule shells (each capsule with 0.2 g of the mixed powder), and finished nucleotide probiotic capsule products were obtained.

### Example 2

A nucleotide probiotic capsule for delaying aging included an elliptical capsule shell and contents. The material of the capsule shell was maltodextrin and soy protein isolate with a weight ratio of 1:1.5. The components of the contents included a mixture of four 5′-mononucleotides or sodium salts thereof and five lyophilized probiotic live bacteria powder, with ratios according to parts by weight as follows: 15 parts of 5′-cytidine monophosphate, 25 parts of 5′-adenosine monophosphate, 8 parts of 5′-disodium uridylate, 10 parts of 5′-disodium guanylate, 6 parts of Lactobacillus acidophilus, 6 parts of Lactobacillus plantarum, 6 parts of Lactobacillus casei, 4 parts of Lactobacillus helveticus, and 3 parts of Pediococcus pentosaceus. The preparation method comprises the following steps: the above-mentioned contents were uniformly mixed according to the ratios to obtain a mixed powder, the mixed powder was loaded into capsule shells (each capsule with 0.2 g of the mixed powder), and finished nucleotide probiotic capsule products were obtained.

### Example 3

A nucleotide probiotic capsule for delaying aging included an elliptical capsule shell and contents. The material of the capsule shell was maltodextrin and soy protein isolate with a weight ratio of 1:2. The components of the contents included a mixture of four 5′-mononucleotides or sodium salts thereof and five lyophilized probiotic live bacteria powder, with ratios according to parts by weight as follows: 20 parts of 5′-cytidine monophosphate, 30 parts of 5′-adenosine monophosphate, 10 parts of 5′-disodium uridylate, 15 parts of 5′-disodium guanylate, 7 parts of Lactobacillus acidophilus, 7 parts of Lactobacillus plantarum, 7 parts of Lactobacillus casei, 5 parts of Lactobacillus helveticus, and 4 parts of Pediococcus pentosaceus. The preparation method comprises the following steps: the above-mentioned contents were uniformly mixed according to the ratios to obtain a mixed powder, the mixed powder was loaded into capsule shells (each capsule with 0.2 g of the mixed powder), and finished nucleotide probiotic capsule products were obtained.

### Example 4

In order to verify the effect of the nucleotide probiotic capsule of the present invention on delaying aging, the anti-oxidant performance of the compound nucleotide is firstly investigated in this example.

A nucleotide mixture of 5′-cytidine monophosphate, 5′-adenosine monophosphate, 5′-disodium uridylate and 5′-disodium guanylate with a weight ratio of 10:20:5:10 was prepared according to the nucleotide probiotic capsule in Example 1. 0.1 g of the nucleotide mixture was dissolved in 100 mL of deionized water, and a nucleotide mixture mother solution was obtained for determining the DPPH free radical scavenging rate and the ABTS free radical scavenging rate.

### 1. Determination of DPPH Free Radical Scavenging Rate

The mother solution was diluted 100, 50, 25, 10 and 1 times to prepare gradient diluents with concentrations of 10 μg/mL, 20 μg/mL, 40 μg/mL, 100 μg/mL, and 500 μg/mL.

150 μL of each of the gradient diluents with the above concentrations was mixed evenly with 150 μL of DPPH solution with a concentration of 60 mg/L (diluted with anhydrous ethanol), placed in a 96-well plate and stored at 30° C. away from light for 30 minutes, and then the absorbance value at 519 nm was measured with a microplate reader. The formula of the DPPH free radical scavenging rate is as follows:

$$DPPH free scavenging rate ( \% ) = \frac{{A}_{Blank} - {A}_{Sample}}{{A}_{Blank}} \times 100$$

In the formula, A_{Blank} represents the absorbance of 150 μL of deionized water mixed with 150 μL of DPPH solution, and Aₛₐₘₚₗₑ represents the absorbance of 150 μL of gradient diluent mixed with 150 μL of DPPH solution. IC₅₀ represents the concentration of a sample when the DPPH free radical scavenging rate reaches 50%.

### 2. Determination of ABTS Free Radical Scavenging Rate

(1) The mother solution was diluted 100, 50, 25, 10 and 1 times to prepare gradient diluents with concentrations of 10 μg/mL, 20 μg/mL, 40 μg/mL, 100 μg/mL, and 500 μg/mL.

(2) 7 mmol/L of ABTS solution was mixed with 140 mmol/L potassium persulfate solution at a ratio of 62.5:1 to obtain a mixed solution, and the mixed solution was allowed to stand overnight away from light at room temperature to obtain an ABTS stock solution, which was diluted to 5 mmol/L of ABTS working solution with deionized water before use.

(3) 0.15 mL of each of the gradient diluents was mixed with 2.85 mL of the ABTS working solution, and was left to stand at 30° C. for 8 minutes, and then the absorbance was measured at 734 nm with deionized water as a blank control.

The formula of ABTS free radical scavenging rate is as follows:

$$ABTS free radical scavenging rate ( \% ) = \frac{{A}_{Blank} - {A}_{Sample}}{{A}_{Blank}} \times 100$$

In the formula, A_{Blank} represents the absorbance of 0.15 mL of deionized water mixed with 2.85 mL of ABTS working solution, and Aₛₐₘₚₗₑ represents the absorbance of 0.15 mL of gradient diluent mixed with 2.85 mL of ABTS working solution. IC₅₀ represents the concentration of a sample when the ABTS free radical scavenging rate reaches 50%.

The anti-oxidant performances of the nucleotide mixture according to the nucleotide probiotic capsules in Examples 2 and 3 were measured with reference to the above same determination methods. The nucleotide mixture was prepared by 5′-cytidine monophosphate, 5′-adenosine monophosphate, 5′-disodium uridylate and 5′-disodium guanylate with a weight ratio of 15:25:8:10 according to Example 2, and the nucleotide mixture was prepared with 5′-cytidine monophosphate, 5′-adenosine monophosphate, 5′-disodium uridylate and 5′-disodium guanylate with a weight ratio of 20:30:10:15 according to Example 3. At the same time, a nucleotide mixture prepared by 5′-cytidine monophosphate, 5′-adenosine monophosphate, 5′-disodium uridylate and 5′-disodium guanylate with a weight ratio of 1:1:1:1 was used as a control experiment. Experimental data are represented by mean±standard deviation, and experimental results are shown in Table 1.

TABLE 1
Anti-oxidant performance of nucleotide mixture

| | Control group | Example 1 | Example 2 | Example 3 |
|---|---|---|---|---|
| IC₅₀(μg/mL, | 185 ± 20 | 126 ± 15 | 119 ± 37 | 103 ± 28 |
| DPPH free radical | | | | |
| scavenging | | | | |
| experiment) | | | | |
| IC₅₀(μg/mL, | 327 ± 29 | 202 ± 36 | 193 ± 29 | 168 ± 43 |
| ABTS free radical | | | | |
| scavenging | | | | |
| experiment) | | | | |

The results showed that, compared with the control group, the nucleotide mixtures of Examples 1 to 3 had relatively high anti-oxidant performance, which can more effectively scavenge free radicals of the body, thereby efficiently delaying aging of the body and enhancing cell vitality.

### Example 5

Body metabolism will generate a large number of oxide free radicals, which destroy cell functions and lead to organ aging, resulting in aging of the body. Superoxide dismutase (SOD) can scavenge superoxide anion free radicals which are harmful to the body. Glutathione peroxidase (GSH-Px) is an important peroxide decomposing enzyme widely existing in the body, which can reduce toxic peroxides to non-toxic hydroxyl compounds, thereby protecting the structure and function of cell membrane from interference and damage by peroxides. Therefore, the activities of SOD and GSH-Px can reflect the anti-oxidant capacity of tissues, and are important indicators for evaluating aging of the organism. MDA is a lipid peroxidation product, which is closely related to the generation of free radicals in the body and the lipid peroxidation degree in body tissues and cells, and can reflect the damage state of oxidative stress in body tissues and is also one of the important indexes for evaluating aging. In order to further verify the effect of the nucleotide probiotic capsule of the present invention on delaying aging, animal experiments are carried out on the nucleotide probiotic capsules prepared in Examples 1 to 3. SAMP-8 mice with rapid aging tendency are used to test the delaying effect of the nucleotide probiotic capsule of the present invention on aging of mice.

First, the nucleotide probiotic capsule prepared in Example 1 was taken as an example for the experiment. 40 mice with rapid aging tendency (SAMP-8 mice commercially obtained from Beijing Vital River Laboratory Animal Technology Co., Ltd.) with body weight of (20±5) g were randomly divided into 4 groups with 10 mice in each group, that is, blank control group (capsules containing only basic feed), control group I (capsules containing only nucleotide mixture with the same amount and the same ratio as those of the nucleotide probiotic capsule of Example 1), control group II (capsules containing only probiotics with the same amount and the same ratio as those of the nucleotide probiotic capsule of Example 1), and experimental group (nucleotide probiotic capsule of Example 1). The animal feed of American Institute of Nutrition (AIN-93G) was used as the basic feed to adaptively feed the mice for one week. The mice in the blank control group, the control groups and the test group were feed corresponding capsules. Each SAMP-8 mouse was given 2 capsules each time, with three times a day for 30 days continuously. The states of the mice were recorded every day.

It can be seen from the results that, with the extension of time, the mice in the blank control group and the control groups I and II showed poor mental state and slow activity, showing signs of aging, while the mice in the test group fed with the nucleotide probiotic capsules of the present invention showed good mental and health conditions.

After the last administration, blood was taken from the fundus oculi of each the mice and was left at room temperature the blood was completely coagulated, and then serum was centrifuged from the coagulated blood for later use. Then the mouse was killed by dislocation, the liver was rapidly separated and was rinsing with sodium chloride solution to prepare 10% of tissue homogenate; the 10% of tissue homogenate was centrifuged to obtain the tissue homogenate supernatant for later use.

The activities of SOD and GSH-Px in serum and liver tissues of mouse were determined according to the kit instructions of superoxide dismutase (SOD) and glutathione peroxidase (GSH-Px), and the contents of malondialdehyde (MDA) in the serum and liver tissues were determined by thiobarbonic acid spectrophotometry. Experimental data are represented by mean±standard deviation, and experimental results are shown in Tables 2 to 4.

TABLE 2
MDA, SOD and GSH-Px contents in mice after feeding the capsules of Example 1

| | | Blank control | | | |
|---|---|---|---|---|---|
| | | group | Control group I | Control group II | Test group |
| MDA content | Serum | 7.02 ± 0.55 | 5.65 ± 0.47 | 6.43 ± 0.51 | 4.19 ± 0.44 |
| (mmol/mL) | Liver | 3.18 ± 0.36 | 2.41 ± 0.20 | 2.98 ± 0.40 | 1.55 ± 0.18 |
| SOD content | Serum | 95 ± 19 | 130 ± 10 | 106 ± 8 | 169 ± 10 |
| (U/mL) | Liver | 87 ± 13 | 119 ± 22 | 99 ± 17 | 148 ± 15 |
| GSH-Px | Serum | 16 ± 3 | 49 ± 7 | 20 ± 6 | 65 ± 6 |
| content | Liver | 21 ± 5 | 60 ± 10 | 30 ± 9 | 88 ± 11 |
| (U/mL) | | | | | |

TABLE 3
MDA, SOD and GSH-Px contents in mice after feeding the capsules of Example 2

| | | Blank control | | | |
|---|---|---|---|---|---|
| | | group | Control group I | Control group II | Test group |
| MDA content | Serum | 7.02 ± 0.55 | 5.27 ± 0.30 | 6.39 ± 0.29 | 4.00 ± 0.23 |
| (mmol/mL) | Liver | 3.18 ± 0.36 | 2.19 ± 0.45 | 2.92 ± 0.40 | 1.40 ± 0.39 |
| SOD content | Serum | 95 ± 19 | 149 ± 23 | 109 ± 16 | 203 ± 24 |
| (U/mL) | Liver | 87 ± 13 | 132 ± 19 | 110 ± 20 | 189 ± 18 |
| GSH-Px | Serum | 16 ± 3 | 55 ± 12 | 23 ± 9 | 73 ± 15 |
| content | Liver | 21 ± 5 | 68 ± 4 | 28 ± 12 | 105 ± 9 |
| (U/mL) | | | | | |

TABLE 4
MDA, SOD and GSH-Px contents in mice after feeding the capsules of Example 3

| | | Blank control | | | |
|---|---|---|---|---|---|
| | | group | Control group I | Control group II | Test group |
| MDA content | Serum | 7.02 ± 0.55 | 5.10 ± 0.65 | 6.25 ± 0.47 | 3.69 ± 0.44 |
| (mmol/mL) | Liver | 3.18 ± 0.36 | 2.13 ± 0.32 | 2.86 ± 0.28 | 1.35 ± 0.18 |
| SOD content | Serum | 95 ± 19 | 155 ± 11 | 113 ± 14 | 215 ± 30 |
| (U/mL) | Liver | 87 ± 13 | 137 ± 25 | 108 ± 29 | 202 ± 10 |
| GSH-Px | Serum | 16 ± 3 | 59 ± 7 | 26 ± 5 | 85 ± 20 |
| content | Liver | 21 ± 5 | 76 ± 10 | 30 ± 13 | 110 ± 5 |
| (U/mL) | | | | | |

The results showed that, compared with the blank control group, the activities of SOD and GSH-Px in the serum and liver tissues of the control groups I and II increased, and the MDA content in serum and liver tissues reduced. Each index value of the test group was obviously better than those of the control groups I and II, further indicating that the anti-aging nucleotide probiotic capsule of the present invention can synergistically enhance the ability of scavenging free radicals of the body, reduce the degree of tissue cells injury, significantly delay the body aging, and has no adverse reactions.

### Example 6

Test Method: 500 cases of female volunteers with aging skin were invited at the same time in the same area, aged 45-60 years old, with an average age of 54 years old.

Selection criteria: the volunteers have mild or moderate skin wrinkles, dry skin, decreased elasticity, pigmentation, sallow complexion and the like. The volunteers were randomly divided into 10 groups of 50 people each, which respectively correspond to a blank control group (capsules containing only equal amount of glucose), a control group 1 (capsules containing only the same amount and the same ratio of nucleotides as those of the nucleotide probiotic capsule of Embodiment 1), a control group 2 (capsules containing only the same amount and the same ratio of probiotics as those of the nucleotide probiotic capsule of Embodiment 1), a control group 3 (capsules containing only the same amount and the same ratio of nucleotides as those of the nucleotide probiotic capsule of Embodiment 2), a control group 4 (capsules containing only the same amount and the same ratio of probiotics as those of the nucleotide probiotic capsule of Embodiment 2), a control group 5 (capsules containing only the same amount and the same ratio of nucleotides as those of the nucleotide probiotic capsule of Embodiment 3), a control group 6 (capsules containing only the same amount and the same ratio of probiotics as those of the nucleotide probiotic capsule of Embodiment 3), a test group 1 (the nucleotide probiotic capsule in Embodiment 1), a test group 2 (the nucleotide probiotic capsule of Embodiment 2), a test group 3 (the nucleotide probiotic capsule of Embodiment 3). Volunteers in each group took the corresponding test capsule as the only anti-aging and anti-oxidant product, and each person took two capsules each time and three times a day, for four weeks. After the test, the volunteers in the test gave feedback on the skin changes and the use feeling. The effect evaluation results are shown in Table 5.

TABLE 5
Effect evaluation results of different groups

| | Number of | Compact | | Memory | Sleep quality |
|---|---|---|---|---|---|
| | volunteers | facial | Full of | improvement/ | improvement/ |
| Group | per group | skin/person | energy/person | person | person |
| Test | 50 | 46 | 45 | 40 | 42 |
| group 1 | | | | | |
| Test | 50 | 45 | 50 | 41 | 46 |
| group 2 | | | | | |
| Test | 50 | 49 | 47 | 43 | 47 |
| group 3 | | | | | |
| Control | 50 | 34 | 30 | 32 | 33 |
| group1 | | | | | |
| Control | 50 | 15 | 20 | 18 | 21 |
| group 2 | | | | | |
| Control | 50 | 37 | 34 | 30 | 38 |
| group 3 | | | | | |
| Control | 50 | 20 | 23 | 25 | 22 |
| group 4 | | | | | |
| Control | 50 | 39 | 35 | 37 | 36 |
| group 5 | | | | | |
| Control | 50 | 25 | 24 | 26 | 25 |
| group6 | | | | | |
| Blank | 50 | 7 | 13 | 10 | 8 |
| control | | | | | |
| group | | | | | |

The above results show that, compared with the blank control group and the control groups 1 to 6, the nucleotide probiotic capsule of the present invention can more effectively improve facial skin, energy, memory, and sleep quality, thereby achieving the purpose of delaying aging.

For those skilled in the art, without departing from the scope of the technical solution of the present invention, many possible changes and modifications can be made to the technical solution of the present invention by using the technical contents disclosed above, or modified into equivalent embodiments with equivalent changes. Therefore, any simple modification, equivalent change and modification made to the above embodiments according to the technical essence of the present disclosure without departing from the technical solution of the present invention shall still belong to the protection scope of the technical solution of the present invention.

1. A nucleotide probiotic capsule for delaying aging, comprising a capsule shell and contents, wherein the contents comprise a mixture of four exogenous nucleotides or sodium salts thereof and five probiotics, with a weight ratio of 45-75 parts of the nucleotide mixture and 20-30 parts of probiotics.

2. The nucleotide probiotic capsule according to claim 1, wherein weight percentages of the four exogenous nucleotides in the contents are as follows: 15-45% of CMP, 15-55% of AMP, 6-30% of UMP, and 14-35% of GMP.

3. The nucleotide probiotic capsule according to claim 1, wherein mass percentages of the various probiotics are as follows: 17-32% of Lactobacillus acidophilus, 17-32% of Lactobacillus plantarum, 17-32% of Lactobacillus casei, 10-23% of Lactobacillus helveticus, and 7-19% of Pediococcus pentosaceus.

4. The nucleotide probiotic capsule according to claim 1, wherein the weight ratios of the contents are as follows: 10-20 parts of 5′-cytidine monophosphate (5′-CMP), 20-30 parts of 5′-adenosine monophosphate (5′-AMP), 5-10 parts of 5′-disodium uridylate (5′-UMPNa₂), 10-15 parts of 5′-disodium guanylate (5′-GMPNa₂), 5-7 parts of Lactobacillus acidophilus, 5-7 parts of Lactobacillus plantarum, 5-7 parts of Lactobacillus casei, 3-5 parts of Lactobacillus helveticus, and 2-4 parts of Pediococcus pentosaceus.

5. The nucleotide probiotic capsule according to claim 4, wherein the weight ratios of the contents are as follows:
10 parts of 5′-cytidine monophosphate, 20 parts of 5′-adenosine monophosphate, 5 parts of 5′-disodium uridylate, 10 parts of 5′-disodium guanylate, 5 parts of Lactobacillus acidophilus, 5 parts of Lactobacillus plantarum, 5 parts of Lactobacillus casei, 3 parts of Lactobacillus helveticus, and 2 parts of Pediococcus pentosaceus;
alternatively, the weight ratios of the contents are as follows:
15 parts of 5′-cytidine monophosphate, 25 parts of 5′-adenosine monophosphate, 8 parts of 5′-disodium uridylate, 10 parts of 5′-disodium guanylate, 6 parts of Lactobacillus acidophilus, 6 parts of Lactobacillus plantarum, 6 parts of Lactobacillus casei, 4 parts of Lactobacillus helveticus and 3 parts of Pediococcus pentosaceus; and
alternatively, the weight ratios of the contents are as follows:
20 parts of 5′-cytidine monophosphate, 30 parts of 5′-adenosine monophosphate, 10 parts of 5′-disodium uridylate, 15 parts of 5′-disodium guanylate, 7 parts of Lactobacillus acidophilus, 7 parts of Lactobacillus plantarum, 7 parts of Lactobacillus casei, 5 parts of Lactobacillus helveticus and 4 parts of Pediococcus pentosaceus.

6. The nucleotide probiotic capsule according to claim 1, wherein a viable bacteria count of each of the probiotics is greater than 1×10⁷ CFU/g.

7. The nucleotide probiotic capsule according to claim 1, wherein a material of the capsule shell is maltodextrin and soy protein isolate with a weight ratio of 1:1-1:2.

8. The nucleotide probiotic capsule according to claim 7, wherein the weight ratio of the maltodextrin to the soy protein isolate is 1:1.5-1:2.

9. A method for preparing the nucleotide probiotic capsule according to claim 1, comprising weighing the nucleotides and the probiotics according to the ratio, then uniformly mixing them to obtain a mixed powder, and loading the mixed powder into a capsule shell to obtain the nucleotide probiotic capsule.

10. An application of the nucleotide probiotic capsule according to claim 1 in preparing health care foods and/or drugs for delaying aging.
